(19) United States
(12) Patent Application Publication
(10) Pub. No.: US 2023/0125495 A1
(43) Pub. Date: Apr. 27, 2023

(54) MINI PERISTALTIC PUMP

(71) Applicant: CareFusion 303, Inc., San Diego, CA (US)

(72) Inventors: Mohammed Mehtab KHAN, Bengaluru (IN); Gene E. Mason, La Habra Heights, CA (US)

(21) Appl. No.: 17/888,149

(22) Filed: Aug. 15, 2022

Related U.S. Application Data
(60) Provisional application No. 63/271,550, filed on Oct. 25, 2021.

(51) Int. Cl. F04B 43/12

(52) U.S. Cl. CPC F04B 43/1276

(57) ABSTRACT

A mini peristaltic pump is provided that includes a base plate, first and second gears disposed on a first side of the base plate, first and second roller wheels disposed on a second side of the base plate and coupled to the first and second gears, respectively, and one or more rollers coupled to each of the first and second roller wheels. A motor is coupled to the first gear, the motor configured to rotate the first gear and the first roller wheel to move the one or more rollers coupled to the first roller wheel in a first orbital path to peristaltically engage and compress a hand pump disposed between the first and second roller wheels. A profile feature on the base may be provided instead of the second gear and roller wheel. Systems and methods are also provided.

### TECHNICAL FIELD

This application claims the benefit of priority under 35 U.S.C. § 119 to U.S. Provisional Patent Application Ser. No. 63/271,550, entitled “MINI PERISTALTIC PUMP,” filed on Oct. 25, 2021, the entire contents of which is incorporated herein by reference.

### TECHNICAL FIELD

The present disclosure generally relates to intravenous (IV) sets, in particular to pumps for IV blood sets.

### BACKGROUND

Medical treatments often include the infusion of a medical fluid (e.g., blood, plasma) to patients using an IV catheter that is connected though an arrangement of flexible tubing and fittings, commonly referred to as an “IV set,” to a source of fluid, for example, an IV blood bag. During operation, medical fluid may be required quickly at greatly increased flow rates. Typical IV blood sets use a manual hand pump bulb or flexible container that is squeezed by hand to rapidly increase fluid flow rate, resulting in muscle fatigue.

For these reasons, it is desirable to provide a pump that mechanically squeezes the flexible container to eliminate muscle fatigue.

### SUMMARY

In one or more embodiments, a mini peristaltic pump includes a base plate, first and second gears disposed on a first side of the base plate, first and second roller wheels disposed on a second side of the base plate and coupled to the first and second gears, respectively, and one or more rollers coupled to each of the first and second roller wheels. A motor is coupled to the first gear, the motor configured to rotate the first gear and the first roller wheel to move the one or more rollers coupled to the first roller wheel in a first orbital path to peristaltically engage and compress a hand pump disposed between the first and second roller wheels.

In one or more embodiments, a mini peristaltic pump includes a base plate, a motor disposed on a first side of the base plate, a roller wheel disposed on a second side of the base plate and coupled to the motor, one or more rollers coupled to the roller wheel and a profile feature disposed on the base adjacent to the roller wheel.

In one or more embodiments, a method of operating a mini peristaltic pump includes placing a hand pump of an intravenous set adjacent one or more roller wheels of a mini peristaltic pump, operating a motor coupled to one of the one or more roller wheels to rotate the one or more roller wheels, rotating one or more rollers coupled to the one or more roller wheels in an orbital pattern, and peristaltically engaging and compressing the hand pump by the one or more rollers to replace manual squeezing of the hand pump by a user, thereby reducing user hand fatigue from the hand pump and increasing fluid flow rate to reduce fluid transfusion time.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure. The motor is configured to rotate the roller wheel to move the one or more rollers coupled to the roller wheel in an orbital path to peristaltically engage and compress a hand pump disposed between the roller wheel and the profile feature.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set with a hand pump.

FIG. 3 depicts a front view of a mini peristaltic pump, according to aspects of the disclosure.

FIG. 4 depicts a rear view of the mini peristaltic pump of FIG. 3, according to aspects of the disclosure.

FIG. 5 depicts a side view of the mini peristaltic pump of FIG. 3, according to aspects of the disclosure.

FIG. 6 depicts a partial rear view of the mini peristaltic pump of FIG. 3, according to aspects of the disclosure.

FIG. 7 depicts a front view of a mini peristaltic pump, according to aspects of the disclosure.

FIG. 8 depicts a rear view of the mini peristaltic pump of FIG. 7, according to aspects of the disclosure.

FIG. 9 depicts a front view of a mini peristaltic pump, according to aspects of the disclosure.

FIG. 10 depicts a front view of a mini peristaltic pump, according to aspects of the disclosure.

FIG. 11 depicts a perspective view of a mini peristaltic pump and IV blood set connected to a patient, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

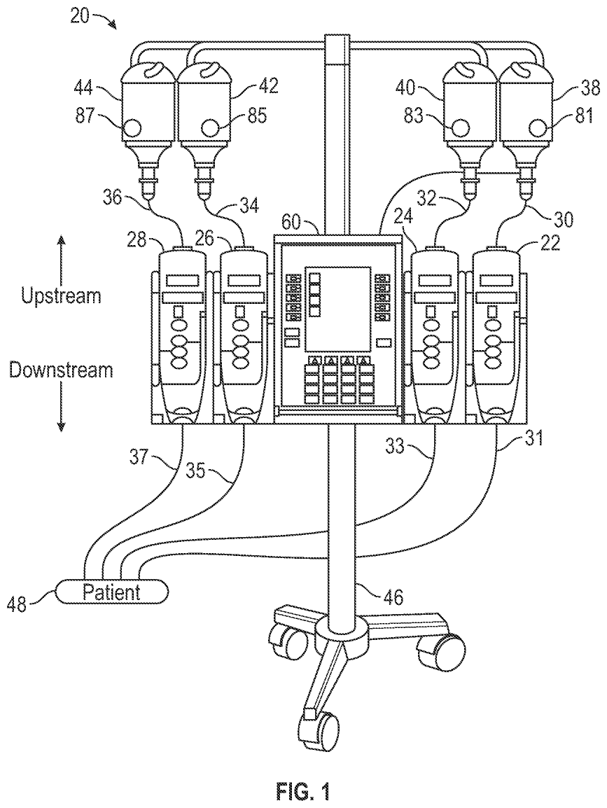

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

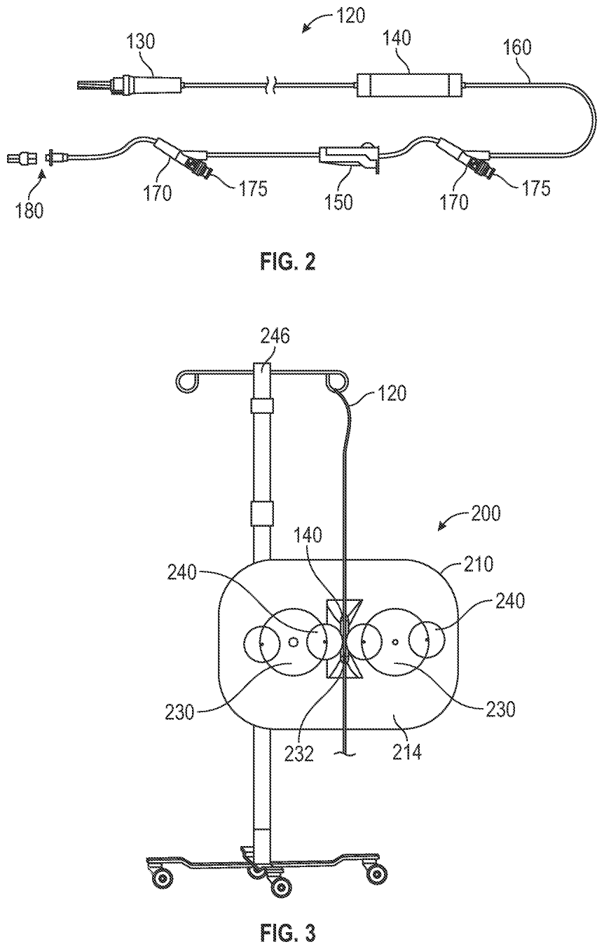

Typically, medical fluid administration sets have more parts than are shown in FIG. 1, such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a hand pump 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. The IV set 120 may include additional infusion components and may be formed of any combination of components and the tubing 160.

In aspects of the disclosure, an electric, reusable pole-mounted peristaltic pump may be mounted behind a blood set hand pump to pump in the blood without hand fatigue. The pump frequency and corresponding flow rate may be chosen by the clinician. Here, the hand pump may be inserted into the pump or the pump may be attached to the hand pump so that hand pump is positioned between rollers that then squeeze the hand pump to cause the pumping action. In aspects of the disclosure, the hand pump may be squeezed between rollers from both sides. In aspects of the disclosure, the hand pump may be squeezed between rollers on one side and a profile feature on one side. In aspects of the disclosure, a reusable mini peristaltic pump may be configured as a benchtop device. In aspects of the disclosure, a reusable mini peristaltic pump may be configured to hang directly from an IV set.

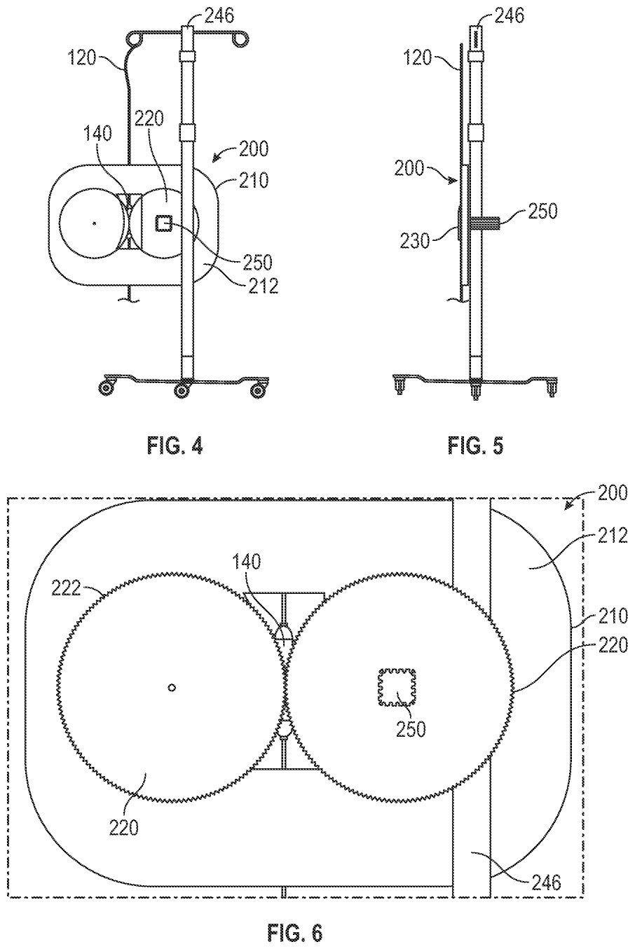

As shown in FIGS. 3-6, a mini peristaltic pump 200 is provided according to aspects of the disclosure. Mini peristaltic pump 200 includes a base plate 210, two gears 220 disposed on a rear side 212 of the base plate 210 and two corresponding roller wheels 230 coupled to the gears 220, with the roller wheels 230 disposed on a front side 214 of the base plate 210. One or more rollers 240 are coupled to each roller wheel 230.

A motor 250 is coupled to one of the gears 220, where rotational movement of the motor 250 causes the coupled gear 220 to rotate. Each gear 220 has multiple teeth 222 and the gears 220 are positioned such that the teeth 222 of each gear 220 are intermeshed, thus causing the second gear 220 not directly coupled to the motor 250 to rotate when the first gear 220 is rotated by the motor 250. In aspects of the disclosure, there may be two motors 250 with each gear 220 coupled to one motor 250.

In aspects of the disclosure, the mini peristaltic pump 200 has an open frame configuration as shown in FIGS. 3-6. In aspects of the disclosure, the mini peristaltic pump 200 may include an enclosure housing some or all of the above-listed elements. In aspects of the disclosure, such an enclosure may have a door or openable portion, providing for opening the mini peristaltic pump 200 to insert or place a hand pump (e.g., hand pump 140) inside.

In aspects of the disclosure, the mini peristaltic pump 200 may include any suitable fastener to mount the mini peristaltic pump 200 to an IV pole 246. For example, the fastener may be a bracket, a hangar, a hook, Velcro®, adhesive, and/or any other suitable fastener.

In use, a hand pump (e.g., hand pump 140) of an IV set (e.g., IV set 120) is inserted or placed into the mini peristaltic pump 200 mounted to IV pole 246, the hand pump 140 being positioned between the roller wheels 230. The IV set 120 is coupled to a fluid container (e.g., fluid supply 38) containing a medical fluid (e.g., blood). In an unactuated state, the mini peristaltic pump 200 may not contact and/or compress the hand pump 140, thus allowing the blood to flow through the IV set 120 at a rate set by a flow controller (e.g., roller clamp 150).

In an actuated state, the motor 250 causes the gears 220 to rotate, which causes the corresponding roller wheels 230 to rotate and move the rollers 240 around the axis of the roller wheel 230 in an orbital manner. The orbiting movement of the rollers 240 causes the rollers 240 to briefly engage and compress the hand pump 140, thus squeezing the hand pump 140 between the two sets of rollers 240 in a peristaltic pumping manner. This peristaltic action squeezes the hand pump 140 to create local pressure gradients that create a fluid flow continuum. The flow rate can be increased by increasing the speed of the motor 250, thereby rotating the rollers 240 faster to squeeze the hand pump 140 more frequently.

In aspects of the disclosure, the roller wheels 230 may be positionally adjustable on the base plate 210, providing for adjusting the size of the gap 232 between the roller wheels 230. Thus, the mini peristaltic pump 200 may be adjusted to work with different sized and/or shaped hand pumps, bulbs, pumpable drip chambers, and the like. In aspects of the disclosure, the mini peristaltic pump 200 is reusable while the IV set 120 is disposable. Accordingly, the mini peristaltic pump 200 may be cleaned as necessary or per medical cleaning protocols.

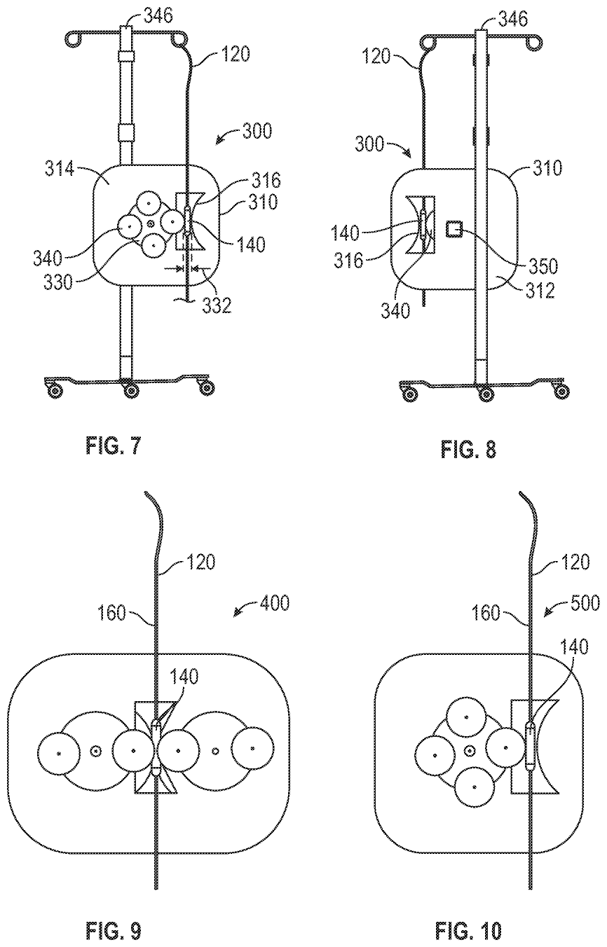

As shown in FIGS. 7 and 8, a mini peristaltic pump 300 is provided according to aspects of the disclosure. Mini peristaltic pump 300 includes a base plate 310, a motor 350 disposed on a rear side 312 of the base plate 310 and a corresponding roller wheel 330 coupled to the motor 350, with the roller wheel 330 disposed on a front side 314 of the base plate 310. One or more rollers 340 are coupled to the roller wheel 330. The base plate 310 includes a profile feature 316 (e.g., protrusion, curved edge) disposed adjacent to the roller wheel 330.

Rotational movement of the motor 350 causes the coupled roller wheel 330 to rotate, thus causing the rollers 340 to rotate as well. The rollers 340 are disposed around the periphery of the roller wheel 330 such that rotation of the roller wheel 330 causes the rollers 340 to move around the axis of the roller wheel 330 in an orbital manner.

In aspects of the disclosure, the mini peristaltic pump 300 has an open frame configuration as shown in FIGS. 7 and 8. In aspects of the disclosure, the mini peristaltic pump 300 may include an enclosure housing some or all of the above-listed elements. In aspects of the disclosure, such an enclosure may have a door or openable portion, providing for opening the mini peristaltic pump 300 to insert or place a hand pump (e.g., hand pump 140) inside.

In aspects of the disclosure, the mini peristaltic pump 300 may include any suitable fastener to mount the mini peristaltic pump 300 to an IV pole 346. For example, the fastener may be a bracket, a hangar, a hook, Velcro®, adhesive, and/or any other suitable fastener.

In use, a hand pump (e.g., hand pump 140) of an IV set (e.g., IV set 120) is inserted or placed into the mini peristaltic pump 300 mounted to IV pole 346, the hand pump 140 being positioned between the roller wheel 330 and the profile feature 316. The IV set 120 is coupled to a fluid container (e.g., fluid supply 38) containing a medical fluid (e.g., blood). In an unactuated state, the mini peristaltic pump 300 may not contact and/or compress the hand pump 140, thus allowing the blood to flow through the IV set 120 at a rate set by a flow controller (e.g., roller clamp 150).

In an actuated state, the motor 350 causes the roller wheel 330 to rotate and move the rollers 340 around the axis of the roller wheel 330 in an orbital manner. The orbiting movement of the rollers 340 causes the rollers 340 to briefly engage and compress the hand pump 140, thus squeezing the hand pump 140 between the rollers 340 and the static profile feature 316 in a peristaltic pumping manner. This peristaltic action squeezes the hand pump 140 to create local pressure gradients that create a fluid flow continuum. The flow rate can be increased by increasing the speed of the motor 350, thereby rotating the rollers 340 faster to squeeze the hand pump 140 more frequently.

In aspects of the disclosure, one or both of the roller wheel 330 and the profile feature 316 may be positionally adjustable on the base plate 310, providing for adjusting the size of the gap 332 between the roller wheel 330 and the profile feature 316. Thus, the mini peristaltic pump 300 may be adjusted to work with different sized and/or shaped hand pumps, bulbs, pumpable drip chambers, and the like. In aspects of the disclosure, the mini peristaltic pump 300 is reusable while the IV set 120 is disposable. Accordingly, the mini peristaltic pump 300 may be cleaned as necessary or per medical cleaning protocols.

As shown in FIG. 9, a mini peristaltic pump 400 may be a self-contained device configured to hang from an IV set, such as IV set 120. The mini peristaltic pump 400 may have many or most all of the features of mini peristaltic pump 200, with a difference being that the fastener is configured to couple the mini peristaltic pump 400 directly to IV set 120 instead of to an IV pole. For example, the fastener may couple the mini peristaltic pump 400 to IV tubing 160 above and/or below the hand pump 140, such that the mini peristaltic pump 400 hangs from the IV tubing 160. As another example, the mini peristaltic pump 400 may be configured to couple and hang directly from just the hand pump 140. Accordingly, the mini peristaltic pump 400 may be small and lightweight so as not to put undue strain on the IV set 120.

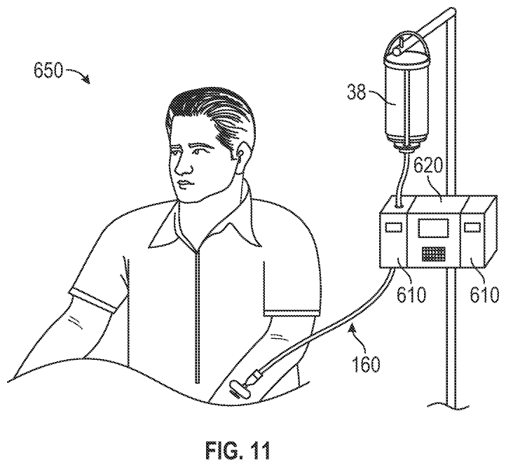

As shown in FIG. 10, a mini peristaltic pump 500 may be a self-contained device configured to hang from an IV set, such as IV set 120. The mini peristaltic pump 500 may have many or most all of the features of mini peristaltic pump 300, with a difference being that the fastener is configured to couple the mini peristaltic pump 500 directly to IV set 120 instead of to an IV pole. For example, the fastener may couple the mini peristaltic pump 500 to IV tubing 160 above and/or below the hand pump 140, such that the mini peristaltic pump 500 hangs from the IV tubing 160. As another example, the mini peristaltic pump 500 may be configured to couple and hang directly from just the hand pump 140. Accordingly, the mini peristaltic pump 500 may be small and lightweight so as not to put undue strain on the IV set 120.

As shown in FIG. 11, a mini peristaltic pump system 600 may be provided. The mini peristaltic pump system 600 may include two mini peristaltic pumps 610 and a controller 620. The mini peristaltic pump 610 may be configured similarly to mini peristaltic pump 200 or mini peristaltic pump 300. Here, a hand pump (not shown) may be inserted into a mini peristaltic pump 610 and the mini peristaltic pump 610 may be set up and/or controlled by the controller 620. Thus, the desired flow rate of blood from the fluid source 38 may be delivered through the IV tubing 160 into the patient by controlling the speed of the moving elements of the mini peristaltic pump 600.

In one or more embodiments of the disclosure, a mini peristaltic pump comprises: a base plate; first and second gears disposed on a first side of the base plate; first and second roller wheels disposed on a second side of the base plate and coupled to the first and second gears, respectively; one or more rollers coupled to each of the first and second roller wheels; and a motor coupled to the first gear, the motor configured to rotate the first gear and the first roller wheel to move the one or more rollers coupled to the first roller wheel in a first orbital path to peristaltically engage and compress a hand pump disposed between the first and second roller wheels.

In aspects of the disclosure, the first and second gears each include multiple teeth, the teeth of the first gear intermeshed with the teeth of the second gear, wherein the rotation of the first gear is configured to cause rotation of the second gear and the second roller wheel to move the one or more rollers coupled to the second roller wheel in a second orbital path to peristaltically engage and compress the hand pump. In aspects of the disclosure, a second motor is coupled to the second gear, the second motor configured to rotate the second gear and the second roller wheel to move the one or more rollers coupled to the second roller wheel in a second orbital path to peristaltically engage and compress the hand pump. In aspects of the disclosure, an enclosure encloses one or more of the base plate, the first and second gears, the first and second roller wheels, the rollers and the motor. In aspects of the disclosure, the enclosure comprises an openable portion configured to provide access for placement of the hand pump inside the enclosure.

In aspects of the disclosure, a fastener is configured to mount the mini peristaltic pump on an intravenous pole. In aspects of the disclosure, one of the first and second roller wheels is positionally adjustable on the base plate, wherein adjustment of the position adjusts a size of a gap between the first and second roller wheels. In aspects of the disclosure, a change in motor speed causes a change in a frequency of hand pump compressions by the one or more rollers. In aspects of the disclosure, the mini peristaltic pump is configured to couple to and hang from intravenous tubing. In aspects of the disclosure, the mini peristaltic pump is configured to couple to and hang from the hand pump.

In one or more embodiments of the disclosure, a mini peristaltic pump comprises: a base plate; a motor disposed on a first side of the base plate; a roller wheel disposed on a second side of the base plate and coupled to the motor; one or more rollers coupled to the roller wheel; and a profile feature disposed on the base adjacent to the roller wheel, wherein the motor is configured to rotate the roller wheel to move the one or more rollers coupled to the roller wheel in an orbital path to peristaltically engage and compress a hand pump disposed between the roller wheel and the profile feature.

In aspects of the disclosure, an enclosure encloses one or more of the base plate, the motor, the roller wheel, the rollers and the profile feature. In aspects of the disclosure, the enclosure comprises an openable portion configured to provide access for placement of the hand pump inside the enclosure. In aspects of the disclosure, a fastener is configured to mount the mini peristaltic pump on an intravenous pole. In aspects of the disclosure, one of the roller wheel and the profile feature is positionally adjustable on the base plate, wherein adjustment of the position adjusts a size of a gap between the roller wheel and the profile feature. In aspects of the disclosure, a change in motor speed causes a change in a frequency of hand pump compressions by the one or more rollers. In aspects of the disclosure, the mini peristaltic pump is configured to one of hang from intravenous tubing and hang from the hand pump.

In one or more embodiments of the disclosure, a mini peristaltic pump system comprises: a mini peristaltic pump; a controller operationally coupled to the mini peristaltic pump; a fluid source fluidically coupled to the mini peristaltic pump via intravenous tubing; and a hand pump coupled to the intravenous tubing and disposed within the mini peristaltic pump, wherein the controller is configured to control a speed of orbital rotation of the one or more rollers to provide a set flow rate of fluid from the fluid source through the intravenous tubing downstream of the mini peristaltic pump.

In one or more embodiments of the disclosure, a method of operating a mini peristaltic pump comprises: placing a hand pump of an intravenous set adjacent one or more roller wheels of a mini peristaltic pump; operating a motor coupled to one of the one or more roller wheels to rotate the one or more roller wheels; rotating one or more rollers coupled to the one or more roller wheels in an orbital pattern; and peristaltically engaging and compressing the hand pump by the one or more rollers to replace manual squeezing of the hand pump by a user, thereby reducing user hand fatigue from the hand pump and increasing fluid flow rate to reduce fluid transfusion time.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A mini peristaltic pump comprising:
a base plate;
first and second gears disposed on a first side of the base plate;
first and second roller wheels disposed on a second side of the base plate and coupled to the first and second gears, respectively;
one or more rollers coupled to each of the first and second roller wheels; and
a motor coupled to the first gear, the motor configured to rotate the first gear and the first roller wheel to move the one or more rollers coupled to the first roller wheel in a first orbital path to peristaltically engage and compress a hand pump disposed between the first and second roller wheels.

2. The mini peristaltic pump of claim 1, wherein the first and second gears each include multiple teeth, the teeth of the first gear intermeshed with the teeth of the second gear, wherein the rotation of the first gear is configured to cause rotation of the second gear and the second roller wheel to move the one or more rollers coupled to the second roller wheel in a second orbital path to peristaltically engage and compress the hand pump.

3. The mini peristaltic pump of claim 1, further comprising a second motor coupled to the second gear, the second motor configured to rotate the second gear and the second roller wheel to move the one or more rollers coupled to the second roller wheel in a second orbital path to peristaltically engage and compress the hand pump.

4. The mini peristaltic pump of claim 1, further comprising an enclosure enclosing one or more of the base plate, the first and second gears, the first and second roller wheels, the rollers and the motor.

5. The mini peristaltic pump of claim 4, wherein the enclosure comprises an openable portion configured to provide access for placement of the hand pump inside the enclosure.

6. The mini peristaltic pump of claim 1, further comprising a fastener configured to mount the mini peristaltic pump on an intravenous pole.

7. The mini peristaltic pump of claim 1, wherein one of the first and second roller wheels is positionally adjustable on the base plate, wherein adjustment of the position adjusts a size of a gap between the first and second roller wheels.

8. The mini peristaltic pump of claim 1, wherein a change in motor speed causes a change in a frequency of hand pump compressions by the one or more rollers.

9. The mini peristaltic pump of claim 1, wherein the mini peristaltic pump is configured to couple to and hang from intravenous tubing.

10. The mini peristaltic pump of claim 1, wherein the mini peristaltic pump is configured to couple to and hang from the hand pump.

11. A mini peristaltic pump comprising:
a base plate;
a motor disposed on a first side of the base plate;
a roller wheel disposed on a second side of the base plate and coupled to the motor;
one or more rollers coupled to the roller wheel; and
a profile feature disposed on the base adjacent to the roller wheel,
wherein the motor is configured to rotate the roller wheel to move the one or more rollers coupled to the roller wheel in an orbital path to peristaltically engage and compress a hand pump disposed between the roller wheel and the profile feature.

12. The mini peristaltic pump of claim 11, further comprising an enclosure enclosing one or more of the base plate, the motor, the roller wheel, the rollers and the profile feature.

13. The mini peristaltic pump of claim 12, wherein the enclosure comprises an openable portion configured to provide access for placement of the hand pump inside the enclosure.

14. The mini peristaltic pump of claim 11, further comprising a fastener configured to mount the mini peristaltic pump on an intravenous pole.

15. The mini peristaltic pump of claim 11, wherein one of the roller wheel and the profile feature is positionally adjustable on the base plate, wherein adjustment of the position adjusts a size of a gap between the roller wheel and the profile feature.

16. The mini peristaltic pump of claim 11, wherein a change in motor speed causes a change in a frequency of hand pump compressions by the one or more rollers.

17. The mini peristaltic pump of claim 11, wherein the mini peristaltic pump is configured to one of hang from intravenous tubing and hang from the hand pump.

18. A mini peristaltic pump system, comprising:
the mini peristaltic pump of claim 1;
a controller operationally coupled to the mini peristaltic pump;
a fluid source fluidically coupled to the mini peristaltic pump via intravenous tubing; and
a hand pump coupled to the intravenous tubing and disposed within the mini peristaltic pump,
wherein the controller is configured to control a speed of orbital rotation of the one or more rollers to provide a set flow rate of fluid from the fluid source through the intravenous tubing downstream of the mini peristaltic pump.

19. A mini peristaltic pump system, comprising:
the mini peristaltic pump of claim 11;
a controller operationally coupled to the mini peristaltic pump;
a fluid source fluidically coupled to the mini peristaltic pump via intravenous tubing; and
a hand pump coupled to the intravenous tubing and disposed within the mini peristaltic pump,
wherein the controller is configured to control a speed of orbital rotation of the one or more rollers to provide a set flow rate of fluid from the fluid source through the intravenous tubing downstream of the mini peristaltic pump.

20. A method of operating a mini peristaltic pump, the method comprising:
placing a hand pump of an intravenous set adjacent one or more roller wheels of a mini peristaltic pump;
operating a motor coupled to one of the one or more roller wheels to rotate the one or more roller wheels;
rotating one or more rollers coupled to the one or more roller wheels in an orbital pattern; and
peristaltically engaging and compressing the hand pump by the one or more rollers to replace manual squeezing of the hand pump by a user, thereby reducing user hand fatigue from the hand pump and increasing fluid flow rate to reduce fluid transfusion time.
